(19) United States
(12) Patent Application Publication
(10) Pub. No.: US 2021/0378735 A1
(43) Pub. Date: Dec. 9, 2021

(54) PHASED ARRAY RADIOFREQUENCY ABLATION CATHETER AND METHOD OF ITS MANUFACTURE

(71) Applicant: St. Jude Medical, Cardiology Division, Inc., St. Paul, MN (US)

(72) Inventor: Bruce Everling, Eagan, MN (US)

(21) Appl. No.: 17/278,034

(22) PCT Filed: Nov. 1, 2019
(86) PCT No.: PCT/US2019/059400

§ 371 Date: Mar. 19, 2021

Related U.S. Application Data
(60) Provisional application No. 62/760,389, filed on Nov. 13, 2018.

(51) Int. Cl. A61B 18/14

(52) U.S. Cl. CPC A61B 18/1492; A61B 2018/0075; A61B 2018/1467

(57) ABSTRACT

A phased radiofrequency ablation catheter includes at least three radiofrequency electrodes arrayed on the catheter shaft. Each electrode is configured to emit radiofrequency energy. The energy emitted by more centrally-located electrodes within the array is phase-delayed relative to the energy emitted by more peripherally-located electrodes within the array. Thus, the radiofrequency energy emitted by the electrodes sums to a therapeutic maximum at a preset therapeutic distance from the catheter shaft, which in turn corresponds to a desired lesion depth in the tissue being ablated. The phrase-delay can be achieved through the use of electronic delay lines, including capacitors and/or coils. In embodiments of the disclosure, the capacitor is integrally formed with one or more of the electrodes, such as by interposing one or more dielectric layers between conductive layers of the electrode.

### BACKGROUND

This application claims the benefit of U.S. provisional application No. 62/760,389, filed 13 Nov. 2018, which is hereby incorporated by reference as though fully set forth herein.

### BACKGROUND

The instant disclosure relates to catheters for use in medical procedures. In particular, the instant disclosure relates to ablation catheters.

Catheters are used for an ever-growing number of procedures, such as diagnostic, therapeutic, and ablative procedures, to name just a few examples. Typically, the catheter is manipulated through the patient's vasculature and to the intended site, for example, a site within the patient's heart.

A typical electrophysiology catheter includes an elongate shaft and one or more electrodes on the distal end of the shaft. The electrodes may be used for ablation, diagnosis, or the like. Oftentimes, these electrodes include ring electrodes that extend about the entire circumference of the catheter shaft, as well as a tip electrode.

It is well known that atrial fibrillation results from disorganized electrical activity in the heart muscle (myocardium). The surgical maze procedure, which involves the creation of a series of surgical incisions through the atrial myocardium in a preselected pattern, has been developed for treating atrial fibrillation.

As an alternative to the surgical incisions of the maze procedure, transmural ablations of the heart may be used. Such ablations may be performed from within the chambers of the heart (endocardial ablation), using endovascular devices (e.g., catheters), which may be introduced through arteries or veins. Various ablation techniques may be used, including, but not limited to, cryogenic ablation, radiofrequency ablation, laser ablation, ultrasonic ablation, and microwave ablation.

The ablation devices can be used to create elongated transmural lesions—that is, lesions extending through a sufficient thickness of the myocardium to block electrical conduction—forming the boundaries of the conductive corridors such as in the atrial or ventricular myocardium. In some ablation procedures, the practitioner (e.g., physician or electrophysiologist) may desire to deliver ablation therapy at significant depth in tissue (e.g., to create a lesion other than on the immediate tissue surface).

### BRIEF SUMMARY

Disclosed herein is an ablation catheter, including: a catheter shaft; and at least three radiofrequency electrodes disposed on the catheter shaft, the at least three radiofrequency electrodes including at least one central electrode and at least two peripheral electrodes, wherein each of the at least three radiofrequency electrodes is configured to emit radiofrequency energy, and wherein the at least one central electrode is configured to emit radiofrequency energy that is phase-delayed relative to the radiofrequency energy emitted by the at least two peripheral electrodes, such that the radiofrequency energy emitted by the at least three radiofrequency electrodes sums to a therapeutic maximum at a preset therapeutic distance from the catheter shaft corresponding to a desired lesion depth in a tissue to be ablated.

According to aspects of the disclosure, the ablation catheter also includes an electronic delay line conductively coupled to the at least one central electrode, wherein the electronic delay line is configured to delay a radiofrequency energy signal to the at least one central electrode. The electronic delay line can include a capacitor and/or a coil.

In embodiments, the at least three radiofrequency electrodes include at least five radiofrequency electrodes, namely, at least one central electrode, at least two peripheral electrodes, and at least two intermediate electrodes. The at least one central electrode can be configured to emit radiofrequency energy that is phase-delayed relative to the radiofrequency energy emitted by the at least two peripheral electrodes and the radiofrequency energy emitted by the at least two intermediate electrodes. Likewise, the at least two intermediate electrodes can be configured to emit radiofrequency energy that is phase-delayed relative to the radiofrequency energy emitted by the at least two peripheral electrodes. It is contemplated that the at least two intermediate electrodes can have identical phase delays.

In other aspects of the disclosure, the at least one central electrode includes at least one capacitor integrally formed therewith. The at least one capacitor operates to phase-delay the radiofrequency energy emitted by the at least one central electrode relative to the radiofrequency energy emitted by the at least two peripheral electrodes.

Also disclosed herein is a method of manufacturing an ablation catheter. The method includes: forming at least three radiofrequency electrodes on a catheter shaft, the at least three radiofrequency electrodes including at least one central electrode and at least two peripheral electrodes; and conductively coupling a delay line to the at least one central electrode, such that radiofrequency energy emitted by the at least one central electrode is phase-delayed relative to radiofrequency energy emitted by the at least two peripheral electrodes.

The delay line can include a capacitor and/or a coil. Alternatively or additionally, the step of conductively coupling a delay line to the at least one central electrode can include integrally forming the delay line with the at least one central electrode. For instance, a capacitor can be formed within the at least one central electrode by disposing at least one dielectric layer between a plurality of conductive layers of the at least one central electrode.

In aspects of the disclosure, the steps of forming at least three radiofrequency electrodes on a catheter shaft and conductively coupling an electronic delay structure to the at least one central electrode can include: forming a first conductive layer extending along a catheter shaft; reducing a diameter of a central segment of the first conductive layer; forming a dielectric layer over the reduced diameter central segment of the first conductive layer; and forming a second conductive layer over the dielectric layer.

The instant disclosure also provides a method of ablating tissue using an ablation catheter. The ablation catheter includes at least three radiofrequency electrodes, namely including at least one central electrode and at least two peripheral electrodes. The method of ablating tissue, in turn, includes the steps of: causing the at least three radiofrequency electrodes to emit radiofrequency energy, wherein the radiofrequency energy emitted by the at least one central electrode is phase-delayed relative to the radiofrequency energy emitted by the at least two peripheral electrodes, such that the radiofrequency energy emitted by the at least three radiofrequency electrodes sums to a therapeutic maximum at a preset therapeutic distance from the catheter shaft corresponding to a desired lesion depth in the tissue.

It is contemplated that the step of causing the at least three radiofrequency electrodes to emit radiofrequency energy, wherein the radiofrequency energy emitted by the at least one central electrode is phase-delayed relative to the radiofrequency energy emitted by the at least two peripheral electrode, includes delivering a radiofrequency signal to the at least one central electrode through a delay line. The delay line can include a capacitor and/or a coil. Optionally, the capacitor can be integrally formed with the electrode.

The present disclosure also relates to an ablation catheter including: a catheter shaft; at least three radiofrequency electrodes disposed on the catheter shaft, the at least three radiofrequency electrodes including a most distal electrode, a most proximal electrode, and at least one interior electrode between the most distal electrode and the most proximal electrode; and a delay line conductively coupled to the at least one interior electrode such that radiofrequency energy emitted by the at least one interior electrode is phase-delayed relative to radiofrequency energy emitted by the most distal electrode and radiofrequency energy emitted by the most proximal electrode. The delay line can be integrally formed with the at least one interior electrode.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 schematically depicts an electrophysiology catheter and associated systems.

FIG. 2 is a close-up view of the distal region of the catheter shown in FIG. 1.

FIGS. 3 and 4 are schematic illustrations of ablation catheters according to embodiments of the instant disclosure, partially cut away to show certain interior details.

FIGS. 5A through 5G illustrate manufacture of an ablation catheter according to embodiments disclosed herein.

FIG. 6 represents use of an ablation catheter as disclosed herein to create a lesion at a depth in tissue to be ablated, with a portion of the catheter cut away to show certain interior details.

### DETAILED DESCRIPTION

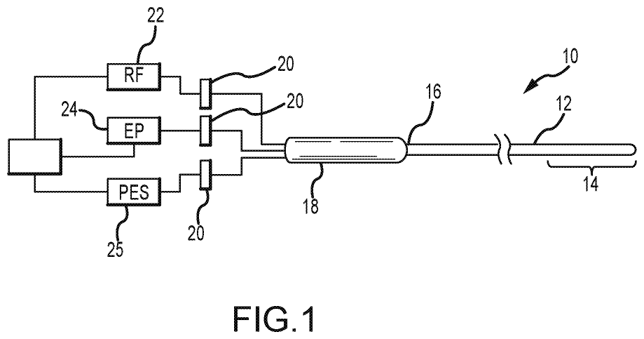

For purposes of illustration, the present teachings will be described in connection with a multi-electrode radiofrequency (RF) ablation catheter 10, such as illustrated in FIG. 1. Those of ordinary skill in the art will appreciate that ablation catheter 10 may be utilized in a cardiac ablation procedure, and aspects of the disclosure will be described herein with reference to a cardiac ablation procedure. It should be understood, however, that the teachings herein can be applied to other forms of RF therapy, including, without limitation, electroporation therapy.

As shown in FIG. 1, catheter 10 generally includes an elongate catheter body (or shaft) 12 having a distal region 14 and a proximal end 16. A handle 18 is shown coupled to proximal end 16. FIG. 1 also shows connectors 20. Connectors 20 are configured to be connected to a source of ablation energy (schematically illustrated as RF source 22, which can be, for example, the Ampere™ RF ablation generator of Abbott Laboratories, Abbott Park, Illinois), an electrophysiology mapping device or system (schematically illustrated as 24, which can be, for example, the EnSite Precision™ cardiac mapping system, also of Abbott Laboratories), and a programmable electrical stimulator (schematically illustrated as 25, which can be, for example the EP-4™ cardiac stimulator, also of Abbott Laboratories). Although FIG. 1 depicts three separate connectors 20, it is within the scope of the instant disclosure to have a combined connector 20 that is configured for connection to two or more of RF source 22, electrophysiology mapping device 24, and programmable electrical stimulator 25.

Various additional aspects of the construction of catheter 10 will be familiar to those of ordinary skill in the art. For example, the person of ordinary skill in the art will recognize that catheter 10 can be made steerable, for example by incorporating an actuator into handle 18 that is coupled to one or more steering wires that extend through elongate catheter body 12 and that terminate in one or more pull rings within distal region 14. Likewise, the ordinarily skilled artisan will appreciate that catheter 10 can be an irrigated catheter, such that it can also be coupled to a suitable supply of irrigation fluid and/or an irrigation pump. As a further example, those of ordinary skill in the art will appreciate that catheter 10 can be equipped with force feedback capabilities.

Insofar as such features are not necessary to an understanding of the instant disclosure, they are neither illustrated in the drawings nor explained in detail herein. By way of example only, however, catheter 10 can incorporate various aspects and features the following catheters, all from Abbott Laboratories: the EnSite™ Array™ catheter; the FlexAbility™ ablation catheter; the Safire™ BLU™ ablation catheter; the Therapy™ Cool Path™ irrigated ablation catheter; the Livewire™ TC ablation catheter; and the TactiCath™ Quartz irrigated ablation catheter.

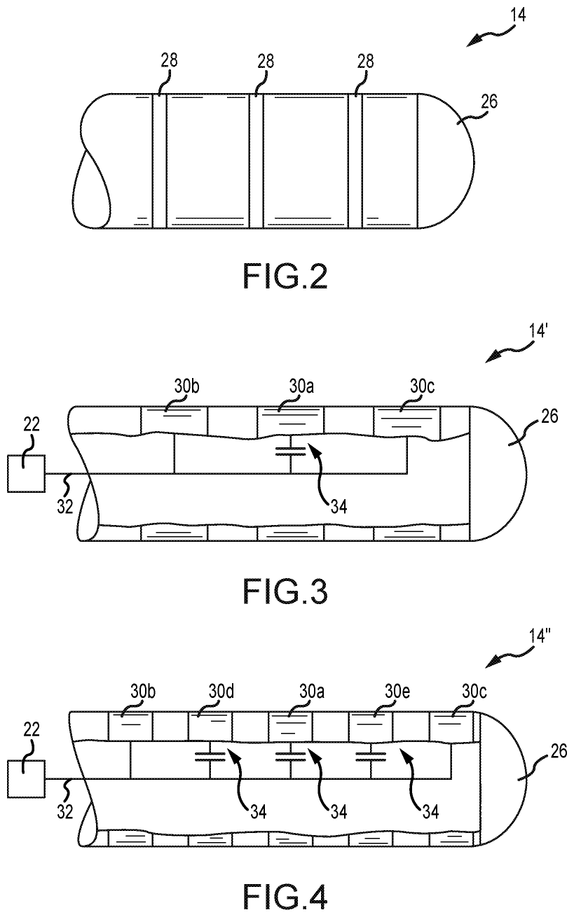

FIG. 2 is a close-up of distal region 14 of catheter 10. Distal region 14 of catheter 10 includes a tip electrode 26 and a plurality of additional electrodes 28 proximal of tip electrode 26. In particular, FIG. 2 depicts three ring electrodes 28. It should be understood, however, that distal region 14 can include any number of electrodes, of any size (e.g. width) and/or inter-electrode spacing, and that such variations are regarded as within the scope of the instant disclosure.

In embodiments of the disclosure, tip electrode 26 and ring electrodes 28 are RF ablation electrodes. That is, tip electrode 26 and ring electrodes 28 are configured to emit RF energy, such as generated by RF source 22, for delivery to a tissue to be ablated as described in further detail below.

FIG. 3 schematically illustrates a first embodiment of an ablation catheter according to the teachings herein. In particular, FIG. 3 depicts distal region 14′ having thereon three radiofrequency electrodes 30, including a central or interior electrode 30a and two peripheral electrodes 30b (a most proximal electrode) and 30c (a most distal electrode).

As used herein, the terms “central electrode,” “interior electrode,” and “peripheral electrode” refer to the relative relationships between electrodes 30 along the length of distal region 14 of catheter 10. In particular, the term “interior electrode” refers to an electrode that has one or more electrodes positioned more distally therefrom along the length of distal region 14 of catheter 10 and one or more electrodes positioned more proximally therefrom along the length of distal region 14 of catheter 10. The term “central electrode” is a special case of interior electrode that has an equal number of electrodes positioned more proximally therefrom and more distally therefrom along the length of distal region 14 of catheter 10. Relative to an interior electrode, the more distally and more proximally positioned electrodes are referred to herein as “peripheral electrodes.” An electrode need not be the most proximally- or most distally-positioned electrode along the length of distal region 14 of catheter 10 to be considered a “peripheral electrode,” although the term “intermediate electrode” will generally be used herein to refer to a peripheral electrode that is positioned between the central electrode and the most distal or most proximal electrode.

Electrodes 30 are coupled to RF source 22 via a conductor 32. Central electrode 30a, however, is configured to emit RF energy that is phase-delayed relative to the RF energy emitted by peripheral electrodes 30b, 30c. Thus, a delay line 34, which may include a capacitor and/or a coil, may be interposed between RF source 22 and central electrode 30a.

FIG. 4 schematically illustrates a second embodiment of an ablation catheter according to the teachings herein. In particular, FIG. 4 depicts distal region 14″ having thereon five radiofrequency electrodes 30, including a central (interior) electrode 30a, two peripheral electrodes 30b (most proximal) and 30c (most distal), and two intermediate electrodes 30d, 30e.

Similar to FIG. 3, electrodes 30 in FIG. 4 are coupled to RF source 22 via a conductor 32. Intermediate electrodes 30d, 30e, however, are configured to emit RF energy that is phase-delayed relative to the RF energy emitted by peripheral electrodes 30b, 30c. This phase-delay can be achieved by interposing a delay line 34, which may include a capacitor and/or a coil, between RF source 22 and intermediate electrodes 30d, 30e. In embodiments of the disclosure, the phase-delay of intermediate electrodes 30d, 30e may be identical (e.g., the capacitance of the capacitor coupled to intermediate electrode 30d may be equal to the capacitance of the capacitor coupled to intermediate electrode 30e).

Likewise, central electrode 30a is configured to emit RF energy that is phase-delayed relative to the RF energy emitted by intermediate electrodes 30d, 30e (and thus also relative to the RF energy emitted by peripheral electrodes 30b, 30c). Again, this phase-delay can be achieved by interposing a delay line 34, which may include a capacitor and/or a coil, between RF source 22 and central electrode 30a. For instance, the capacitance of the capacitor coupled to central electrode 30a may exceed the capacitance of the capacitors coupled to intermediate electrodes 30d, 30e.

In general, it should be understood that the most interior electrode(s) (e.g., 30a) will have the greatest phase delay and the most peripheral electrode(s) (e.g., 30b, 30c) will have the least (and potentially no) phase delay. Intermediate electrode(s) (e.g., 30d, 30e) will have intermediate phase delay.

FIGS. 3 and 4 illustrate a common conductor 32 coupled to each electrode 30 (e.g., a one-to-many correspondence), with delay line(s) 34 interposed between conductor 32 and certain electrodes 30. It should be understood, however, that multiple conductors 32 can be used (e.g., a one-to-one correspondence between conductors 32 and electrodes 30).

Likewise, it should be understood that delay line(s) 34 need not be positioned within the interior of distal region 14 proximate electrodes 30, but rather need only be interposed between RF source 22 and electrode(s) 30. For instance, in some aspects of the disclosure, delay line(s) 34 can be located elsewhere within catheter shaft 12, within handle 18, or entirely external to catheter 10 (e.g., within connector 20 that couples catheter 10 to RF source 22).

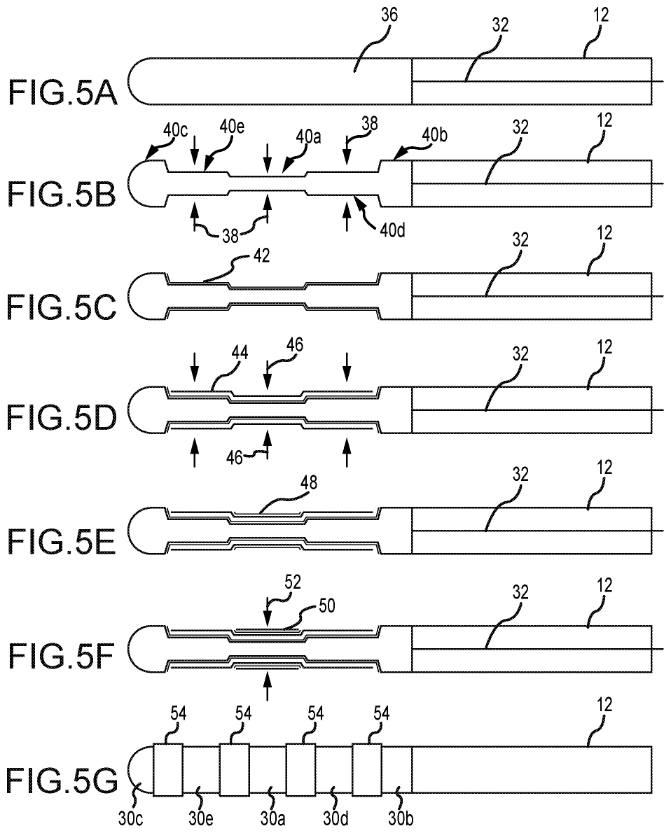

A method of manufacturing the ablation catheter schematically illustrated in FIG. 4 can be understood with reference to FIGS. 5A-5G. FIG. 5A depicts a catheter shaft 12 having a single conductive electrode 36 thereon.

In FIG. 5B, the diameter of electrode 36 is modified, such as by swaging (arrows 38), to form five regions 40a-40e. As described below, regions 40a-40e ultimately correspond to electrodes 30a-30e, as described above with reference to FIG. 4.

In FIG. 5C, a dielectric layer 42 is formed over regions 40a, 40d, and 40e of electrode 36.

In FIG. 5D, a conductive layer 44 is formed over dielectric layer 42. Conductive layer 44 can also be swaged (arrows 46) to fit.

In FIG. 5E, an additional dielectric layer 48 is formed over conductive layer 44 in region 40a.

In FIG. 5F, a further conductive layer 50 is formed over dielectric layer 48. Conductive layer 50 can be swaged (arrows 52) to fit.

In FIG. 5G, insulators 54 can be added, thereby defining electrodes 30a-30e.

Electrodes 30b and 30c are electrically connected to the same point, and thus will be in phase with each other. Electrodes 30d and 30e, which each include one capacitor integrally-formed (e.g., by electrode 36, dielectric layer 42, and conductive layer 44), are phase-delayed relative to electrodes 30b and 30c, but in phase with each other. Electrode 30a, which includes two capacitors integrally-formed (e.g., by electrode 36, dielectric layer 42, conductive layer 44, dielectric layer 48, and conductive layer 50), is phase-delayed relative to electrodes 30d and 30e, and thus also phase-delayed relative to electrodes 30b and 30c.

In use, ablation catheter 10 is coupled (e.g., via connector(s) 20) to RF source 22, which generates an RF signal that travels to electrodes 30 (e.g., via conductor 32). Delay line(s) 34 operate to introduce a delay in the RF signal as delivered to the more interior electrodes (e.g., electrodes 30a, 30d, and 30e) as compared to the more peripheral electrodes (e.g., electrodes 30b, 30c).

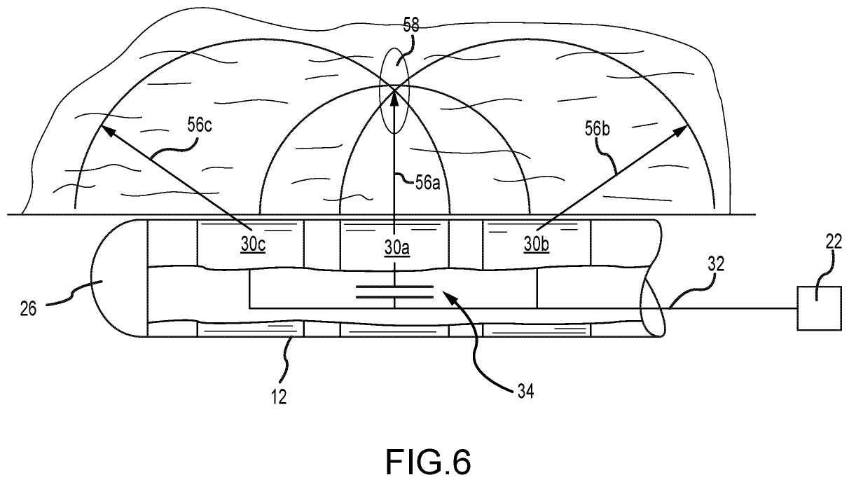

Because of the phase delays introduced by delay line(s) 34, and as illustrated in FIG. 6, the RF energy 56a, 56b, 56c respectively emitted by electrodes 30a, 30b, 30c sums to a therapeutic maximum within the tissue adjacent to the catheter, where the arriving RF energy 56a, 56b, 56c is in phase, at a preset therapeutic distance 58 from catheter shaft 12, corresponding to a desired lesion depth in the tissue to be ablated. Conversely, at other locations within tissue (e.g., where the arriving RF energy is not in phase), less tissue damage will occur.

Those of ordinary skill in the art will recognize that therapeutic distance 58 will vary by application. In embodiments of the disclosure, however, therapeutic distance 58 is between about 3 mm and about 5 mm into the tissue. In other embodiments of the disclosure, therapeutic distance 58 is even deeper, which desirably allows for the creation of lesions on the epicardial surface via an endocardial device.

Moreover, because the lesion is created via a summation of multiple, phased RF wavefronts, each individual wavefront can be lower energy. This also limits tissue damage at locations where the practitioner does not desire to create a lesion, such as on the tissue surface immediately adjacent ablation catheter 10.

In other words, aspects of the disclosure allow a practitioner to create an ablation lesion at depth in tissue without modifying the RF source and with minimized risk of tissue damage at locations other than the intended lesion location.

Although several embodiments of this invention have been described above with a certain degree of particularity, those skilled in the art could make numerous alterations to the disclosed embodiments without departing from the spirit or scope of this invention.

For example, although three-electrode and five-electrode ablation catheter embodiments are described above, those of ordinary skill in the art would understand how to extend the teachings herein to ablation catheters having different numbers of electrodes.

As another example, although linear catheters are described above, those of ordinary skill in the art would understand how to extend the teachings herein to ablation catheters having other shapes (e.g., hoop catheters, spiral catheters, and so forth).

As another example, those of ordinary skill in the art would appreciate how to adapt the foregoing teachings to create lesions at different tissue depths/distances from the ablation catheter.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. An ablation catheter comprising:
a catheter shaft; and
at least three radiofrequency electrodes disposed on the catheter shaft, the at least three radiofrequency electrodes comprising at least one central electrode and at least two peripheral electrodes,
wherein each of the at least three radiofrequency electrodes is configured to emit radiofrequency energy, and
wherein the at least one central electrode is configured to emit radiofrequency energy that is phase-delayed relative to the radiofrequency energy emitted by the at least two peripheral electrodes, such that the radiofrequency energy emitted by the at least three radiofrequency electrodes sums to a therapeutic maximum at a preset therapeutic distance from the catheter shaft corresponding to a desired lesion depth in a tissue to be ablated.

2. The ablation catheter according to claim 1, further comprising an electronic delay line conductively coupled to the at least one central electrode, wherein the electronic delay line is configured to delay a radiofrequency energy signal to the at least one central electrode.

3. The ablation catheter according to claim 2, wherein the electronic delay line comprises a capacitor.

4. The ablation catheter according to claim 2, wherein the electronic delay line comprises a coil.

5. The ablation catheter according to claim 1, wherein:
the at least three radiofrequency electrodes comprise at least five radiofrequency electrodes, the at least five radiofrequency electrodes comprising at least one central electrode, at least two peripheral electrodes, and at least two intermediate electrodes,
the at least one central electrode is configured to emit radiofrequency energy that is phase-delayed relative to the radiofrequency energy emitted by the at least two peripheral electrodes and the radiofrequency energy emitted by the at least two intermediate electrodes, and
the at least two intermediate electrodes are configured to emit radiofrequency energy that is phase-delayed relative to the radiofrequency energy emitted by the at least two peripheral electrodes.

6. The ablation catheter according to claim 5, wherein the at least two intermediate electrodes have identical phase delays.

7. The ablation catheter according to claim 1, wherein the at least one central electrode comprises at least one capacitor integrally formed therewith, wherein the at least one capacitor operates to phase-delay the radiofrequency energy emitted by the at least one central electrode relative to the radiofrequency energy emitted by the at least two peripheral electrodes.

8. A method of manufacturing an ablation catheter, the method comprising:
forming at least three radiofrequency electrodes on a catheter shaft, the at least three radiofrequency electrodes comprising at least one central electrode and at least two peripheral electrodes;
conductively coupling a delay line to the at least one central electrode, such that radiofrequency energy emitted by the at least one central electrode is phase-delayed relative to radiofrequency energy emitted by the at least two peripheral electrodes.

9. The method according to claim 8, wherein the delay line comprises a capacitor.

10. The method according to claim 8, wherein the delay line comprises a coil.

11. The method according to claim 8, wherein the step of conductively coupling a delay line to the at least one central electrode comprises integrally forming the delay line with the at least one central electrode.

12. The method according to claim 11, wherein the step of integrally forming the delay line with the at least one central electrode comprises forming a capacitor within the at least one central electrode by disposing at least one dielectric layer between a plurality of conductive layers of the at least one central electrode.

13. The method according to claim 8, wherein the steps of forming at least three radiofrequency electrodes on a catheter shaft and conductively coupling an electronic delay structure to the at least one central electrode comprise:
forming a first conductive layer extending along a catheter shaft;
reducing a diameter of a central segment of the first conductive layer;
forming a dielectric layer over the reduced diameter central segment of the first conductive layer; and
forming a second conductive layer over the dielectric layer.

14. A method of ablating tissue using an ablation catheter including at least three radiofrequency electrodes, the at least three radiofrequency electrodes comprising at least one central electrode and at least two peripheral electrodes, the method comprising:
causing the at least three radiofrequency electrodes to emit radiofrequency energy, wherein the radiofrequency energy emitted by the at least one central electrode is phase-delayed relative to the radiofrequency energy emitted by the at least two peripheral electrodes,
such that the radiofrequency energy emitted by the at least three radiofrequency electrodes sums to a therapeutic maximum at a preset therapeutic distance from the catheter shaft corresponding to a desired lesion depth in the tissue.

15. The method according to claim 14, wherein the step of causing the at least three radiofrequency electrodes to emit radiofrequency energy, wherein the radiofrequency energy emitted by the at least one central electrode is phase-delayed relative to the radiofrequency energy emitted by the at least two peripheral electrode comprises delivering a radiofrequency signal to the at least one central electrode through a delay line.

16. The method according to claim 15, wherein the delay line comprises a capacitor.

17. The method according to claim 16, wherein the capacitor is integrally formed with the at least one central electrode.

18. The method according to claim 15, wherein the delay line comprises a coil.

19. An ablation catheter comprising:
a catheter shaft;
at least three radiofrequency electrodes disposed on the catheter shaft, the at least three radiofrequency electrodes comprising a most distal electrode, a most proximal electrode, and at least one interior electrode between the most distal electrode and the most proximal electrode; and
a delay line conductively coupled to the at least one interior electrode such that radiofrequency energy emitted by the at least one interior electrode is phase-delayed relative to radiofrequency energy emitted by the most distal electrode and radiofrequency energy emitted by the most proximal electrode.

20. The ablation catheter according to claim 19, wherein the delay line is integrally formed with the at least one interior electrode.
